SEQUENCE LISTING (15 sequences)

SEQ ID NO: 1 - DNA, 20 bp, Artificial Sequence
  Synthetic Primer/Probe
taccccagccagtgtcaaca

SEQ ID NO: 2 - DNA, 25 bp, Artificial Sequence
  Synthetic Primer/Probe
tccatgataggctttgatgactttc

SEQ ID NO: 3 - DNA, 26 bp, Artificial Sequence
  Synthetic Primer/Probe
ccggacaaaccaaagacagcctctga

SEQ ID NO: 4 - DNA, 23 bp, Artificial Sequence
  Synthetic Primer/Probe
ctcaactacatggtctacatgtt

SEQ ID NO: 5 - DNA, 23 bp, Artificial Sequence
  Synthetic Primer/Probe
gtagactccacgacatactcagc

SEQ ID NO: 6 - DNA, 25 bp, Artificial Sequence
  Synthetic Primer/Probe
cccatcaccatcttccaggagcgag

SEQ ID NO: 7 - DNA, 20 bp, Artificial Sequence
  Synthetic Primer/Probe
taccccggccagtgtcaaca

SEQ ID NO: 8 - DNA, 25 bp, Artificial Sequence
  Synthetic Primer/Probe
tccatgataggcttggatgactttc

SEQ ID NO: 9 - DNA, 26 bp, Artificial Sequence
  Synthetic Primer/Probe
ccggataaaccaaagacagcatccga

SEQ ID NO: 10 - DNA, 28 bp, Artificial Sequence
  Synthetic Primer/Probe
ctcaactacatggtgtacatgttccagt

SEQ ID NO: 11 - DNA, 25 bp, Artificial Sequence
  Synthetic Primer/Probe
gtggactccacaacatactcagcac

SEQ ID NO: 12 - DNA, 25 bp, Artificial Sequence
  Synthetic Primer/Probe
tccatctccatcttccaggagcgag

SEQ ID NO: 13 - DNA, 24 bp, Artificial Sequence
  Synthetic Primer/Probe
ggacattctggaaatgacagtgaa

SEQ ID NO: 14 - DNA, 17 bp, Artificial Sequence
  Synthetic Primer/Probe
agcgcagccgtcatctg

SEQ ID NO: 15 - DNA, 15 bp, Artificial Sequence
  Synthetic Primer/Probe
cggaacctgcagcgg

(19) United States
(12) Patent Application Publication
(10) Pub. No.: US 2007/0072227 A1
(43) Pub. Date: Mar. 29, 2007

(54) Gamma secretase notch biomarkers

(75) Inventors: Jere E. Meredith, Haddam, CT (US); Craig Thomas Polson, Middletown, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/526,995

(22) Filed: Sep. 26, 2006

Related U.S. Application Data
(60) Provisional application No. 60/720,921, filed on Sep. 27, 2005.

(51) Int. Cl. C12Q 1/68; G01N 33/53; G06F 19/00

(52) U.S. Cl. 435/6; 435/7.92; 702/19; 702/20

(57) ABSTRACT

The present invention relates to biomarker indicators, including polypeptides, polynucleotides and small molecules, that measure γ-secretase mediated Notch processing. These indicators have utility in predicting and/or determining in vivo Notch-related toxicity associated with inhibition of Notch processing mediated by γ-secretase. The reagents and methods of the invention can be utilized before, after, or concurrently with, pre-clinical, clinical, and/or post-clinical testing. The reagents and methods of the invention can be used to identify and maintain preferred doses of test compounds and thereby prevent medical complications, such as GI cellular damage.

This application claims priority to United States Provisional Patent Application Ser. No. 60/720,921, filed Sep. 27, 2005, which is hereby incorporated by reference in its entirety.

### FIELD OF THE INVENTION

The present invention relates generally to gamma secretase and to biomarkers. More specifically, the invention relates to methods for measuring γ-secretase-mediated Notch processing in vivo. Changes in these biomarkers correlate with Notch-related toxicity associated with the modulation of gamma secretase-mediated activity. The invention also relates to employing these biomarkers to identify a preferred dose of a test compound and to the generation of a dosing schedule, which can be employed as part of a therapeutic regimen.

### BACKGROUND OF THE INVENTION

Alzheimer's disease (AD) is a neurodegenerative disorder and the most common form of dementia in the elderly (reviewed in Hardy & Selkoe, (2002) Science 297(5580):353-6; Mattson, (2004) Nature 430(7000):631-9 and Walsh & Selkoe, (2004) Neuron 44(1):181-93). AD is characterized clinically by a progressive loss in cognitive function, including memory impairment, deterioration in language and visuo-spatial functions and alterations in personality and behavior. Pathologically, AD is characterized by the presence of β-amyloid plaques and neurofibrillary tangles in the cortex and hippocampus. Amyloid β peptide (Aβ) is the main component of plaques and tau, the main component of tangles. Genetic evidence from familial early onset forms of AD (FAD) suggests that aggregation and accumulation of Aβ, specifically Aβ1-42, initiates the cascade of events leading to neuropathology and dementia. Further support for the amyloid hypothesis is provided by transgenic mouse models where overproduction of Aβ 1-42 recapitulates many of the hallmarks of AD including formation of plaques and cognitive deficits. Recent evidence from a triple transgenic mouse model of AD suggests that Aβ aggregation and accumulation proceeds and initiates tangle formation (Oddo et al., (2003) Neurobiol. Aging 24(8):1063-70; Oddo et al., (2004) Neuron 43(3):321-32; Oddo et al., (2003) Neuron 39(3):409-21).

Aβ is generated by proteolytic processing of APP by two enzymes, β-amyloid cleavage enzyme (BACE) and gamma secretase (γ-secretase). γ-secretase is a complex comprised of four proteins: presenilin (presenilin-1 or -2 ), nicastrin APH-1 and PEN-2 (De Strooper, (2003) Neuron 38(1):9-12). Presenilin-1 and -2 contain transmembrane aspartyl residues that have been shown to be essential for catalytic processing activity of the complex. The majority of the mutations linked to the early onset, familial form of AD (FAD) are associated with either PS-1 or PS-2. γ-secretase appears to have the capacity to process any type I transmembrane protein that has undergone ectodomain shedding (Struhl & Adachi, (2000) Mol. Cell 6:625-636). In addition to APP, γ-secretase also been shown to cleave a number of other substrates including the Notch family of receptors (1-4), the Notch ligands Delta-1 and Jagged-2, E-Cadherin, ErbB4 and CD44 (De Strooper, (2003) Neuron 38(1):9-12). Genetic evidence indicates that the γ-secretase complex is critically required for Notch signaling and function, at least in context of the developing embryo (Struhl & Greenwald, (1999) Nature (London) 398(6727):522-525; Ye et al., (1999) Nature (London) 398(6727):525-529; Levitan & Greenwald, (1995) Nature (London) 377(6547):351-5; Levitan & Greenwald, (1998) Development (Cambridge, U. K.) 125(18):3599-3606; Huppert et al., (2000) Nature 405:966-970; Donoviel et al., (1999) Genes Dev. 13(21):2801-2810; Herreman et al., (1999) Proc. Natl. Acad. Sci. U.S.A. 96(21):11872-11877). The physiological role of γ-secretase-mediated cleavage of Notch in the adult and of the other substrates is not known.

Notch is an evolutionarily conserved and widely expressed single-span type I transmembrane receptor that plays a prominent role in regulating cell fate decisions in the developing embryo (reviewed in Artavanis-Tsakonas et al., (1999) Science 284(5415):770-6 and Kadesch, (2000) Exp. Cell Res. 260(1):1-8.). The role of Notch in the adult is less clear but Notch proteins are expressed in various adult tissues and are thought to play a role in regulating stem cell differentiation. Four Notch genes have been identified in mammals (Notch 1-4); all four Notch proteins are cleaved by γ-secretase (Mizutani et al., (2001) Proc. Natl. Acad. Sci. U.S.A. 98(16):9026-9031). Notch activation is induced by binding, in trans, to the Delta/Serrate/LAG2 family of transmembrane ligands. Notch signal transduction is mediated by three cleavage events: (a) cleavage at Site 1 in the extracellular domain (Logeat et al., (1998) Proc. Natl. Acad. Sci. U.S.A. 95(14):8108-12); (b) cleavage at Site 2 just N-terminal to the extracellular/transmembrane domain boundary following ligand binding (Brou et al., (2000) Mol. Cell 5(2):207-216; Mumm et al., (2000) Mol. Cell 5(2):197-206; Pan & Rubin, (1997) Cell 90(2):271-80); and (c) cleavage at Site 3 (S3) within the transmembrane near the transmembrane/cytoplasmic domain boundary (Schroeter et al., (1998) Nature (London) 393(6683):382-386; Kopan et al., (1996) Proc. Natl. Acad. Sci. U.S.A. 93(4):1683-8). Site 3 cleavage is required for release of the Notch intracellular domain (NICD) and is mediated by γ-secretase (Struhl & Greenwald, (1999) Nature (London), 398(6727):522-525; Levitan & Greenwald, (1998) Development (Cambridge, U. K.) 125(18):3599-3606; Mizutani et al., (2001) Proc. Natl. Acad. Sci. U.S.A. 98(16):9026-9031; Saxena et al., (2001) J. Biol. Chem. 276(43):40268-73; De Strooper et al., (1999) Nature (London) 398(6727):518-522). NICD activates transcription mediated by the CBF1/Su(H)/LAG-1 family of DNA-binding proteins and induces expression of various genes including HES-1 (Jarriault et al., (1998) Mol. Cell Biol. 18(12):7423-31; Ohtsuka et al., (1999) EMBO J. 18(8):2196-207). NICD-regulated transcription is thought to be a key component of Notch-mediated signal transduction.

The development of γ-secretase inhibitors to block APP cleavage and Aβ generation is one therapeutic approach for the treatment of AD. This approach, however, is beset by the potential for mechanism-based toxicity due to inhibition of Notch processing. Indeed, Notch-related toxicities have been observed in studies where animals have been dosed subchronically with γ-secretase inhibitors (Wong et al., (2004) J. Biol. Chem. 279(13):12876-82; Searfoss et al., (2003) J. Biol. Chem. 278(46):46107-16; Milano et al., (2004) Toxicol. Sci. 82(1):341-58). One toxicity consistently observed following three or more days of treatment is an intestinal goblet cell metaplasia (Wong et al., (2004) J. Biol. Chem. 279(13):12876-82; Searfoss et al., (2003) J. Biol. Chem. 278(46):46107-16; Milano et al., (2004) Toxicol. Sci. 82(1):341-58). This lesion is similar to the phenotype observed in Hes-1 KO mice (Jensen et al., (2000) Nature Genet. 24(1):36-44), suggesting that the inhibitor-induced lesion is linked to inhibition of Notch signaling through Hes-1. In addition to the GI lesion, alterations in lymphocyte development have also been noted after 5-15 days of dosing, including thymus atrophy, reductions in thymocyte numbers and alterations in thymocyte differentiation. These results are also consistent with inhibition of Notch processing and inhibition of it's role in regulating lymphocyte development (Wong et al., (2004) J. Biol. Chem. 279(13):12876-82).

Despite the potential for mechanism-based toxicity, γ-secretase inhibitors have been developed with some or complete specificity for inhibiting APP processing (Petit et al., (2003) J. Neurosci. Res. 74(3):370-7; Weggen et al., (2001) Nature 414(6860):212-6; Barten et al., (2005) J. Pharmacol. Exp. Ther. 312(2):635-43). In order to screen such inhibitors in vivo, it is desirable that biomarkers be developed that can be employed to monitor safety with respect to potential Notch-related toxicities.

A set of indicators that could be used to gauge toxic effects in vivo would therefore be of great value. A single set of reagents and standards could be used to evaluate test compounds from initial screening, through testing in pre-clinical (e.g., drug discovery) species, and potentially in clinical trials. Such universal indicators of toxicity preferably meet several criteria. First, they preferably are able to correctly identify toxic compounds with diverse mechanisms of action, including various chemical classes/chemotypes. Second, changes in these biomarkers are preferably consistent, quantifiable and reflect the degree of toxic insult. Third, assays are preferably adaptable to high throughput technologies without becoming prohibitively expensive. Fourth, in vivo sample collection is preferably non- or minimally invasive, i.e. urine or blood is collected. Fifth, since there may be a need to analyze archival samples, it is preferable that the biomarker is stable.

Thus, what is needed is a method of determining in vivo the ability of a test compound known or suspected to modulate Notch processing mediated by γ-secretase. The present invention solves this and other problems.

### SUMMARY OF THE INVENTION

In one aspect, the present invention provides a method of identifying a modulator of Notch processing in vivo mediated by γ-secretase. In one embodiment, the method comprises (a) determining an amount of an indicator in a sample comprising leukocytes acquired from a query subject in the presence and absence of the test compound; and (b) comparing the amount of indicator acquired from the query subject in the presence of the test compound with an amount of indicator acquired from the query subject in the absence of the test compound; wherein a change in the amount of indicator acquired in the presence of the test compound, compared with the amount of indicator acquired in absence of the test compound, indicates the compound modulates Notch processing mediated by γ-secretase activity.

In the context of the method, the indicator can be selected from the group consisting of a Notch-regulated transcription factor, a membrane protein and a Notch-regulated secreted factor. The Notch-regulated transcription factor can be selected from the group consisting of Hes-1 and TCF3. The membrane protein can be selected from the group consisting of SLC11A1, CD14, TRL4. The Notch-regulated secreted factor can be selected from the group consisting of CSPG and IL10. In one embodiment, the leukocytes are lymphocytes. In another embodiment the leukocytes are T cells. The query subject can be selected from the group consisting of mice, rats, dogs, guinea pigs and humans.

In one embodiment, the step of determining the amount of the indicator can comprise determining an amount of mRNA encoding the indicator present in the sample. In another embodiment the step of determining the amount of the indicator comprises determining an amount of indicator protein present in the sample. The indicator amounts can be determined by employing an analytical technique selected from the group consisting of Western blot, ELISA, RIA, quantitative real-time PCR, fluorescence activated cell sorting (FACs) and immunohistochemistry. The method can, but need not, be employed in a high-throughput operation. The method can further comprise repeating the method for each of a plurality of different test compounds. Additionally, the method can be performed in a clinical trial.

In another aspect, the present invention provides a method of identifying a preferred dose of a test compound known or suspected to modulate Notch processing in vivo mediated by γ-secretase. In one embodiment, the method comprises (a) determining an amount of an indicator in a sample comprising leukocytes acquired from a query subject in the absence of the test compound; (b) determining an amount of indicator and a Notch-related toxicity level in a sample comprising leukocytes acquired from a query subject in the presence of a first dose of the compound; (c) repeating step (b) a for a plurality of different test compound doses; (d) comparing (i) the indicator amount; and (ii) the Notch-related toxicity acquired in the presence of two or more doses of the test compound; and (e) identifying a preferred dose of a compound known or suspected to modulate Notch processing mediated by γ-secretase based on an analysis of the comparison.

In the context of the method, the indicator can be selected from the group consisting of a Notch-regulated transcription factor, a membrane protein and a Notch-regulated secreted factor. The Notch-regulated transcription factor can be selected from the group consisting of Hes-1 and TCF3. The membrane protein can be selected from the group consisting of SLC11A1, CD14, TRL4. The Notch-regulated secreted factor can be selected from the group consisting of CSPG and IL10. In one embodiment, the leukocytes are lymphocytes. In another embodiment the leukocytes are T cells. The query subject can be selected from the group consisting of mice, rats, dogs, guinea pigs and humans.

In one embodiment, the step of determining the amount of the indicator can comprise determining an amount of mRNA encoding the indicator present in the sample. In another embodiment the step of determining the amount of the indicator comprises determining an amount of indicator protein present in the sample. The indicator amounts can be determined by employing an analytical technique selected from the group consisting of Western blot, ELISA, RIA, quantitative real-time PCR, fluorescence activated cell sorting (FACs) and immunohistochemistry. The method can, but need not, be employed in a high-throughput operation. The method can further comprise repeating the method for each of a plurality of different test compounds. Additionally, the method can be performed in a clinical trial. The Notch-related toxicity can be, for example, GI toxicity or goblet cell hyperplasia

In yet another aspect, the present invention provides a method of generating a dosing schedule for a test compound known or suspected to modulate an activity mediated by γ-secretase. In one embodiment the method comprises (a) determining an amount of indicator in a sample comprising leukocytes acquired from a query subject in the absence of the test compound; (b) determining an amount of indicator in a sample comprising leukocytes acquired from the query subject in the presence of a first dose of the test compound at multiple time points; (c) repeating step (b) for one or more doses of the test compound (d) determining the Notch-related toxicity acquired in the presence of two or more doses of the test compound; and (e) generating a dosing schedule based on a comparison of the observed indicator amounts and pharmacodynamics and associated Notch-related toxicity.

In the context of the method, the indicator can be selected from the group consisting of a Notch-regulated transcription factor, a Notch-regulated cell surface receptor and a Notch-regulated secreted factor. The Notch-regulated transcription factor can be selected from the group consisting of Hes-1 and Hes-5. The Notch-regulated cell surface receptor can be selected from the group consisting of Notch-1, Notch-2, Notch-3 and Notch-4. The Notch ligand can be selected from the group consisting of Delta-1, Delta-3, Delta-4, Jagged-i and Jagged-2. The Notch-regulated secreted factor can be IL2. In one embodiment, the leukocytes are lymphocytes. In another embodiment the leukocytes are T cells. The query subject can be selected from the group consisting of mice, rats, dogs, guinea pigs and humans. In one embodiment, the step of determining the amount of the indicator can comprise determining an amount of mRNA encoding the indicator present in the sample. In another embodiment the step of determining the amount of the indicator comprises determining an amount of indicator protein present in the sample. The indicator amounts can be determined by employing an analytical technique selected from the group consisting of Western blot, ELISA, RIA, quantitative real-time PCR, fluorescence activated cell sorting (FACs) and immunohistochemistry. The method can, but need not, be employed in a high-throughput operation. The method can further comprise repeating the method for each of a plurality of different test compounds. Additionally, the method can be performed in a clinical trial. The Notch-related toxicity can be, for example, GI toxicity or goblet cell hyperplasia and can be determined by a technique selected from the group consisting of examining the immunohistochemistry of tissue sections and examining the morphology of goblet cells. The method can further comprising monitoring the dosing schedule.

### BRIEF DESCRIPTION OF THE DRAWINGS

FIG. 1 is a bar graph demonstrating dose-dependent reductions in peripheral WBC Hes-1 mRNA levels observed after treatment with γ-secretase inhibitors.

FIG. 2 is a series of six micrographs demonstrating the observation that γ-secretase inhibitors alter crypt stem cell differentiation in the small intestine.

FIG. 3A is a bar graph depicting a rat GI histopath time course.

FIG. 3B is a bar graph demonstrating that in rats, acute changes in the WBC Hes-1 mRNA levels are predictive of the GI histopathology observed on day 3 and day 4.

### DETAILED DESCRIPTION OF THE INVENTION

In one particular aspect of the present invention, Hes-1, as measured in peripheral white blood cells (WBCs), was shown to be a biomarker for Notch-related toxicity. In another aspect, it is disclosed that Hes-1 is expressed in circulating WBC and that Hes-1 levels can easily be detected using assays with whole blood. Hes-1 can be detected in multiple species including rat, dog and human. As described herein, Hes-1 mRNA levels exhibit dose dependent reductions associated with γ-secretase inhibition. The changes observed for Hes-1 occur acutely following a single dose of compound and the changes observed correlate with the onset of Notch-mediated toxicity, particularly GI toxicity, which develops after 3-4 days of dosing. These results indicate that WBC Hes-1 and other indicators can be used as an acute predictive marker for monitoring and screening for Notch-related toxicity due to γ-secretase inhibition in both animal models and humans.

### I. Definitions

Following long-standing patent law convention, the terms “a” and “an” mean “one or more” when used in this application, including the claims.

As used herein, the term “test compound” means any molecule, chemical entity, composition, drug, therapeutic agent, chemotherapeutic agent, or biological agent known or suspected to be capable of preventing, ameliorating, or treating a disease or other medical condition. The term includes small molecule compounds, antisense reagents, siRNA reagents, antibodies, and the like. A test compound can be assayed in accordance with the methods of the invention at any stage, e.g., during drug discovery or development, clinical trials, during pre-trial testing, or following FDA-approval.

As used herein, the term “about,” when referring to a value or to an amount of mass, weight, time, volume, concentration or percentage is meant to encompass variations of ±20% or ±10%, more preferably ±5%, even more preferably ±1%, and still more preferably ±0.1% from the specified amount, as such variations are appropriate.

As used herein, the term “indicator” means any detectable substance that is known or suspected to be correlated with Notch processing mediated by γ-secretase. An indicator can take any chemical form and can be, for example, mRNA, a protein or protein fragment, a peptide, DNA or a small molecule.

As used herein, the term “Notch processing mediated by γ-secretase” means cleavage of one or more of the Notch family of proteins (1-4) at the S3 cleavage site by the γ-secretase complex

As used herein, the term “Notch-related toxicity” means an undesired and/or deleterious effect that arises as a direct or indirect result of inhibition of Notch processing by γ-secretase. One example of Notch-related toxicity is GI toxicity. Another example of Notch-related toxicity is goblet cell hyperplasia.

As used herein, the term “high throughput” takes its ordinary meaning and refers to an operation in which a plurality of samples are run in a short period of time. For example, welled plates can be employed in a high throughput operation and can facilitate the rapid analysis of a plurality of samples.

### II. Method Of Identifying a Test Compound That Modulates Notch Processing in Vivo Mediated by γ-Secretase

Although γ-secretase modulators can be employed to combat AD, Notch-related toxicities have been observed in studies where animals have been dosed with γ-secretase inhibitors Thus, when a γ-secretase modulator, or suspected modulator, is administered to a subject, the potential for effects on γ-secretase-mediated Notch-processing exists. In certain situations this could lead to Notch-related toxicities. It is desirable, therefore, to identify not only compounds that modulate an activity mediated by γ-secretase, but also to identify the degree of Notch-related toxicity, if any, associated with a given compound. This procedure can be employed in a range of applications, for example as a step in a compound screening process or as a component of a safety profile protocol. Ultimately, the procedure can lead to compounds that are effective γ-secretase modulators, yet also exhibit an acceptable Notch-related toxicity profile. Alternatively, modulation of γ-secretase-mediated Notch processing may itself be the therapeutic goal such as in the treatment of certain cancers. Although Notch processing can proceed by any of a variety of mechanisms, the present invention primarily addresses Notch processing mediated by γ-secretase.

In accordance with the above, in one embodiment of the present invention, a method of identifying a test compound that modulates Notch processing in vivo mediated by γ-secretase is disclosed. In one embodiment, the method comprises (a) determining an amount of an indicator in a sample comprising leukocytes acquired from a query subject in the presence and absence of a test compound; and (b) comparing the amount of indicator acquired from the query subject in the presence of the test compound with the amount of indicator acquired from the query subject in the absence of the test compound, wherein a change in the amount of indicator acquired in the presence of the test compound, compared with the amount of indicator acquired in absence of the test compound, indicates the compound modulates Notch processing mediated by γ-secretase activity.

A test compound employed in the method can be any compound that is known or suspected to modulate Notch processing mediated by γ-secretase. Such a test compound can comprise, but is not limited to, a small molecule. A test compound can also comprise, for example, a protein, which can comprise an antibody, a peptide or a single or double-stranded nucleic acid such as DNA, RNA, an antisense reagent or an RNAi reagent.

Notch processing mediated by γ-secretase refers to cleavage of one or more of the Notch family of proteins at the S3 cleavage site by the γ-secretase complex.

In one step of the method, an amount of an indicator in a sample comprising leukocytes acquired from a query subject is determined in the presence and absence of the test compound. An indicator can be of any form; for example an indicator can be the expressed product of, or mRNA encoding, a gene whose expression is regulated either directly or indirectly by Notch signaling, such as a transcription factor, a cell surface receptor or a secreted factor. Examples of transcription factors include Hes-1 and TCF3. Examples of membrane proteins include SLC11A1, CD14, TRL4. Examples of secreted factors include IL10 and CSPG. Any indicator can be employed, with the proviso that the level of the indicator correlate with the level of Notch processing. In the present invention the term “amount” refers to a quantity of an indicator, and the measurement can be direct (e.g., a quantity of indicator) or indirect (e.g., a measure of fluorescence).

In this and other embodiments of the present invention, a sample comprises leukocytes and is acquired from a query subject. The sample can be, for example, a whole blood sample or a buffy coat fraction of blood, and either sample can be employed. The sample need only comprise leukocytes, examples of which include, but are not limited to, lymphocytes or T-cells.

With further respect to a sample of the present invention, although a sample purification step can form an additional step in this and other the other embodiments of the present invention disclosed herein, such a purification step is optional. Indeed, one advantage of the present invention is that no sample preparation or purification is necessary.

In the present invention, a query subject can be any subject from which a sample comprising leukocytes can be obtained. For example, a query subject can be a human, rat, mouse, dog, or guinea pig.

The amount of indicator is determined in the presence and absence of the test compound. The technique by which the determination is made is dependent, in part, on the nature of the indicator. In one example, when an indicator is a protein such as HES-1, the determination can be made by quantitatively determining the amount of HES-1 protein that is present in the sample. Standard molecular biological techniques can be employed in the quantitation of an indicator protein such as Western blot, ELISA, RIA, fluorescence activated cell sorting (FACS) , immunohistochemistry and immunofluorescence microscopy . These standard analytical and biochemical techniques are well-known to those of ordinary skill in the art, and are described in various references (see, e.g., Sambrook et al., Molecular Cloning: A Laboratory Manual. 3^{rd} ed. Cold Spring Harbor Laboratory Press (2001) and Ausubel et al., Short Protocols in Molecular Biology (Short Protocols in Molecular Biology). 5^{th} ed. Wiley Publishers (2002), both of which are incorporated herein by reference).

In another example, mRNA encoding a protein such as HES-1 can be an indicator; in this case, the determination can be made by quantitating the amount of mRNA encoding the protein that is present in the sample. Standard molecular biological techniques can be employed in the quantitation of mRNA such as quantitative real-time PCR (QRT-PCR) and Northern blotting. These standard analytical and biochemical techniques are well-known to those of ordinary skill in the art, and are described in various references (see, e.g., Sambrook et al., Molecular Cloning: A Laboratory Manual. 3^{rd} ed. Cold Spring Harbor Laboratory Press (2001) and Ausubel et al., Short Protocols in Molecular Biology (Short Protocols in Molecular Biology). 5^{th} ed. Wiley Publishers (2002), both of which are incorporated herein by reference).

Continuing with the instant embodiment, once an amount of indicator present in a sample is determined in the presence and absence of the test compound, the indicator amount acquired from the query subject in the presence of the test compound is compared with an indicator amount acquired from the query subject in the absence of the test compound. A change in the indicator amount acquired in the presence of the test compound, compared with the indicator amount acquired in absence of the test compound, indicates the compound modulates Notch processing mediated by γ-secretase activity.

The comparison can, but need not, comprise a statistical analysis of acquired data (e.g., indicator amounts). A statistical analysis can provide additional information regarding the comparison, such as a confidence interval or margins of error.

The comparison indicates a degree to which a given compound modulates Notch processing mediated by γ-secretase is determined relative to a baseline, which is Notch processing determined in the absence of the test compound. This information can be particularly beneficial to screening protocols. Since the methods of the present invention do not require a sample purification step, although such a step can be performed and may sometimes be desirable, the methods are particularly amendable to inclusion in a high throughput screening process or system, although low throughput usage is also within the scope of the present invention. When the method forms a component of a high-throughput operation, welled plates, such as 96 well plates, can be employed.

When the method is employed in any screening operation, whether high or low throughput, the method can further comprise repeating the method for each of a plurality of different test compounds. In this application, the relative abilities of two or more test compounds in a sample set can be assayed to determine which one or more compounds of the sample set meet a given set of user-defined criteria; such criteria can include an identification of the test compound as the strongest modulator of Notch regulated γ-secretase activity in a panel of test compounds, the weakest modulator of Notch regulated γ-secretase activity in a panel of test compounds, or modulation of Notch regulated γ-secretase activity to a desired degree by a member of a panel of test compounds.

In the context of a screening operation, a user-selected activity cut-off value can be employed. In this context, test compounds exhibiting modulatory activity either above or below the cut-off can be pursued in additional studies, while those that do not meet this criteria are excluded from further characterization. In a further embodiment, the instant method can form a component of a clinical trial.

It is noted that although this embodiment of the present invention has been described in the context of identifying a modulator of Notch processing mediated by γ-secretase activity, the identification of test compounds that are not modulators can be equally valuable information and this forms yet another aspect of the invention.

### III. Method of Identifying a Preferred Dose of a Test Compound Known or Suspected to Modulate Notch Processing in Vivo Mediated by γ-Secretase

Under some conditions, modulation of Notch processing in vivo mediated by γ-secretase activity can result in Notch-related toxicity. More particularly, a given compound may be determined to be a highly effective modulator of γ-secretase activity, but such compounds can have unwanted side effects, including Notch-related toxicity due to inhibition of γ-secretase-mediated Notch processing. Until the present disclosure, those of ordinary skill in the art were unable to rapidly, accurately and conveniently determine the Notch-related toxicity associated with a given compound at a given dose unless such compounds were dosed for multiple days. The present invention solves this problem by providing biomarkers for assessing the level of γ-secretase-mediated Notch processing. These biomarkers are predictive of Notch-related toxicities observed after multiple days of dosing. This ability can facilitate more efficient screening of γ-secretase modulators, with one benefit being the conservation of time and resources. The method offers the further advantage that a profile for a test compound can be generated that includes a measure of an indicator predictive of Notch-related toxicity. A preferred dose of a test compound can be determined to minimize the potential for Notch-related toxicity.

In some cases modulation of Notch processing mediated by γ-secretase may be the desired therapeutic goal. The present invention enables one to rapidly, accurately and conveniently determine compound efficacy by providing a biomarker for assessing the level of γ-secretase-mediated Notch processing.

In one embodiment of a method of identifying a preferred dose of a test compound known or suspected to modulate Notch processing mediated by γ-secretase, comprises determining an amount of indicator in a sample comprising leukocytes acquired from a query subject in the absence of the test compound. As is the case with all the methods of the present invention, an indicator can be a protein, peptide, nucleic acid or even a small molecule.

As described herein, any indicator can be employed in this or the other methods of the present invention. Representative indicators include a transcription factor, a membrane protein and a secreted factor. Examples of transcription factors include Hes-1 and TCF3. Examples of membrane proteins include SLC11A1, CD 14, TRL4. Examples of secreted factors include IL10 and CSPG.

The step of determining an amount of indicator in the sample will depend on the nature of the indicator. For example, the determining can comprise determining an amount of mRNA encoding the indicator that is present in the sample. In another example, when the indicator is a protein, the determining can comprise determining an amount of indicator protein is present in the sample. Standard molecular biological techniques can be employed in the quantitation of mRNA such as quantitative real-time PCR and Northern blotting. Standard molecular biological techniques can be employed in the quantitation of an indicator protein such as Western blot, ELISA, RIA, fluorescence activated cell sorting (FACS), immunohistochemistry, quantitative RT-PCR (QRT-PCR) and immunofluorescence microscopy. These standard analytical and biochemical techniques are well-known to those of ordinary skill in the art, and are described in various references (see, e.g., Sambrook et al., Molecular Cloning: A Laboratory Manual. 3^{rd} ed. Cold Spring Harbor Laboratory Press (2001) and Ausubel et al., Short Protocols in Molecular Biology (Short Protocols in Molecular Biology). 5^{th} ed. Wiley Publishers (2002), both of which are incorporated herein by reference).

The method can further comprise an optional sample purification step, such as the isolation of a subset of WBCs, although no such a purification step is necessary.

A sample of the present invention is acquired from a query subject, which can be any subject from which a sample comprising leukocytes can be obtained. Examples of leukocytes include T-cells or lymphocytes. Representative examples of query subjects of the present invention include humans, rats, mice, dogs, or guinea pigs.

After the sample is acquired, an amount of the indicator, is determined to establish a baseline. Query subjects are then dosed with a test compound at different doses. A second sample is then acquired and the amount of the indicator determined.

The change in the amount of indicator observed between the first and second sample measures the change in γ-secretase-mediated Notch processing. Where appropriate, a measure of Notch-related toxicity, including but not limited to alterations in gastrointestinal and T and B cell differentiation, can be determined in subjects dosed with test compounds for multiple days. Alterations in gastrointestinal differentiation can be assessed by examining tissue sections by immunohistochemical techniques for goblet cell hyperplasia. Alterations in T cell differentiation can be assessed by determining the extent of thymus atrophy and measuring the reduction in thymocyte number.

The Notch-related toxicity associated with a particular test compound at a particular dose can be quantitatively determined to facilitate a numerical comparison with results obtained from other test compounds and/or doses. For example, a scoring scale can be employed to assign numerical values to the amount of Notch-related toxicity that is observed for a given test compound at a given dose.

Alternatively, a qualitative assessment of Notch-related toxicity can be made. Notch-related toxicity, in the context of the present invention, includes, but is not limited to gastrointestinal toxicity. In this approach, tissue sections can be examined for goblet cell hyperplasia and an overall assessment of deviations from a predetermined set of criteria taken as baseline (e.g., “normal”) noted in a non-numerical format, such as a description of the nature, degree, indications and/or extent of deviations from baseline. For example, general descriptions such as “minimal changes in morphology observed” or “significant changes in morphology observed” can be employed. See Table III.

Continuing with this embodiment of the method, the above steps can be repeated for a plurality of different test compound doses. By repeating the steps at different test compound doses, a database of indicator amounts and Notch-related toxicities can be compiled for a range of different test compound doses can be compiled to establish a correlation between the indicator and Notch-related toxicities.

After acquiring data comprising indicator amounts and Notch-related toxicities at different doses, the indicator amounts and Notch-related toxicities acquired in the presence of two or more doses of the test compound are compared. The comparison can take any form and can depend on the nature of the Notch-related toxicity data that is being employed. By way of example, if a quantitative measure of Notch-related toxicity is employed, the toxicities can be numerically compared. If a qualitative measure is being employed, general descriptors can be compared. The results of the comparison can be used to establish the relationship between the level of the indicator and any Notch-related toxicity observed after two or more doses.

Finally, a preferred dose of a compound known or suspected to modulate Notch processing mediated by γ-secretase activity can be identified based on an evaluation of the amount of indicator observed at a given dose of a test compound. In such an analysis, doses at which higher changes in the indicator are observed may be excluded as less preferred, due to the risk of Notch-related toxicities in a test subject or patient. Similarly, doses at which only small changes in the indicator are observed may be preferable to minimize the risk for Notch-related toxicities.

In a related application of the present invention, the described methods can be employed to evaluate each of a plurality of different test compounds. This application can form an element of an overall screening operation or as an element of a protocol for optimizing a particular modulator of an activity mediated by γ-secretase. The method can be employed in a clinical or pre-clinical (e.g., drug discovery) setting.

### IV. Method of Generating a Dosing Schedule for a Test Compound Known or Suspected to Modulate an Activity Mediated by -Secretase

In some situations, it can be desirable to generate a dosing schedule for a modulator. In the context of the present invention, a dosing schedule can be generated in which the potential for developing a Notch-related toxicity is monitored using an indicator as a function of time. This type of dosing schedule can provide guidelines as to when, and how much of, a preferred dose of a test compound, which can be identified as described above, should be administered to a patient. By following a dosing schedule generated as described herein, a physician or researcher administering the test compound can minimize unwanted toxic effects due to Notch-related toxicity.

Accordingly, in yet a further embodiment of the present invention, a method of generating a dosing schedule for a test compound known or suspected to modulate an activity mediated by γ-secretase is provided. In one embodiment of the method, an indicator level in a sample comprising leukocytes acquired from a query subject in the absence of a test compound. Representative indicators include proteins, nucleic acids including mRNA and DNA and small molecules. Representative indicators include a transcription factor, a membrane protein and a secreted factor. Examples of transcription factors include Hes-1 and TCF3. Examples of membrane proteins include SLC11A1, CD14, TRL4. Examples of secreted factors include IL10 and CSPG.

Continuing, an indicator amount in a sample comprising leukocytes acquired from the query subject in the presence of a first dose of the test compound at a first time point is determined. The step of determining an amount of indicator in the sample will depend on the nature of the indicator. For example, the determining can comprise determining an amount of mRNA encoding the indicator that is present in the sample. In another example, when the indicator is a protein, the determining can comprise determining an amount of indicator protein is present in the sample. Standard molecular biological techniques can be employed in the quantitation of mRNA such as quantitative real-time PCR and Northern blotting. Standard molecular biological techniques can be employed in the quantitation of an indicator protein such as Western blot, ELISA, RIA, fluorescence activated cell sorting (FACS), immunohistochemistry, quantitative RT-PCR (QRT-PCR) and immunofluorescence microscopy. These standard analytical and biochemical techniques are well-known to those of ordinary skill in the art, and are described in various references (see, e.g., Sambrook et al., Molecular Cloning: A Laboratory Manual. 3^{rd} ed. Cold Spring Harbor Laboratory Press (2001) and Ausubel et al., Short Protocols in Molecular Biology (Short Protocols in Molecular Biology). 5^{th} ed. Wiley Publishers (2002), both of which are incorporated herein by reference).

In the instant method, a representative sample comprises leukocytes. The leukocytes can be, for example, T cells or lymphocytes. The sample can, therefore, be, for example, a serum sample or a whole blood sample acquired from a query subject. As stated herein, no sample preparation is necessary, although in some cases one or more sample preparation and/or purification steps may be desirable.

An amount of indicator in a sample comprising leukocytes acquired from the query subject in the presence of a first dose of the test compound at a first time point is then determined. The first dose can be selected based on a previous assessment of the efficacy of the test compound. After administering a first dose, the sample can be acquired at a first time point. As is the case with the first dose of the test compound, the first time point can be selected based on prior studies of the test compound. The technique employed for the determining can follow the guidelines described herein, and will depend, in part, on the nature of the indicator.

Continuing, Notch-related toxicity including, but not limited to, alterations in gastrointestinal and T and B cell differentiation can be determined in subjects dosed with test compounds for multiple days. Alterations in gastrointestinal differentiation can be assessed by examining tissue sections by immunohistochemical techniques for goblet cell hyperplasia. Alterations in T cell differentiation can be assessed by determining the extent of thymus atrophy and measuring the reduction in thymocyte number.

The steps of determining an indicator amount at multiple time points following a dose and determining Notch-related toxicity can then be performed for two or more different doses of the test compound. This step can be repeated for any number of test compound doses. By repeating the process at different test compound doses, a database of Notch-related toxicities for different doses and different pharmacokinetic profiles can be compiled.

Finally, a dosing schedule based on the observed indicator amount and Notch-related toxicity associated with a dose and a pharmacokinetic profile of the test compound can be generated. A dosing schedule will take into account Notch-related toxicities associated with different test compound doses and pharmacokinetic profiles. Together, this information can provide a researcher with a profile of a compound's activity at different time points, which can be used to optimize a treatment regimen for a patient.

This embodiment of the present invention can be employed in a high-throughput operation and, in one embodiment of the method, the method can be repeated for each of a plurality of different test compounds. When the method forms a component of a high-throughput operation, welled plates, such as 96 well plates, can be employed.

This embodiment can be performed in a clinical trial. In one aspect of this embodiment, the method further comprises monitoring the dosing schedule so as to minimize toxicity yet maximize an efficacious treatment regimen.

In all embodiments of the present invention, a test compound can be administered using any suitable drug delivery technique known in the art, such as orally or parenterally. The selection of a delivery technique will be a function of the physical and chemical properties of the test compound itself and, consequently, the formulation can dictate appropriate routes of delivery. In one embodiment, a test compound is formulated in accordance with routine procedures as a pharmaceutical composition adapted for intravenous administration to human beings. Typically, test compounds for intravenous administration are solutions in sterile isotonic aqueous buffer. Where necessary, a test compound may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where a test compound is to be administered by infusion, it can be dispensed with an infusion bottle containing sterile pharmaceutical grade water or saline. Where a composition is administered by injection, an ampoule of sterile water for injection or saline can be provided so that the ingredients may be mixed prior to administration.

### EXAMPLES

The following Examples have been included to illustrate various exemplary modes of the invention. Certain aspects of the following Examples are described in terms of techniques and procedures found or contemplated by the inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### Example 1

Whole blood from rat, human, or dog was collected using PAXGENE (Qiagen) blood RNA tubes. Tubes containing whole blood were incubated at room temperature for 2 hours prior to storing at −20° C. until isolation of total RNA.

PAXGENE (Qiagen) blood RNA tubes containing 2.5 ml whole blood were slowly thawed at room temperature for 2 hours followed by isolation of total RNA using the PAXGENE (Qiagen) blood RNA kit. Final RNA was DNAse treated using an on-column DNAse kit (Qiagen). Total RNA was quantitated at a wavelength of 260 nm in 10 mM Tris-HCL pH 7.5 (1 OD unit=44 ng/μl).

Typically 500 ng RNA was used to prepare cDNA using the SUPERSCRIPT III first strand synthesis kit (Invitrogen) with random hexamer primers. The final reaction volume of 21 μl was diluted 1:2 to a total volume of 42 μl, which is 12 ng input RNA/μl.

### PCR Reaction

The conditions and thermocycler settings for the PCR reaction were as follows:

Reaction Mixture

2.5 μl of for and rev primers (9 μM stock)
0.125 μl of probe (40 μM stock)
12.5 μl 2× Taq MASTERMIX (Eurogenetec, available from VWR)
5.375 μl water
2 μl cDNA (which translates to 24 ng input RNA)
   ABI 7700 Thermocycler Cycling Conditions
50° C. 2 min
95° C. 10 min
40 cycles of 95° C. 15 sec/60° C. 1 minute

A standard curve using full length cDNA for HES-1 or GAPDH was run on each plate and final ct values for each of the unknowns were calculated off the standard curve to yield input DNA. Input DNA for the HES-1 reaction was divided by input DNA for the GAPDH (which was included as a control) to give a normalized input DNA. This value was then normalized to either a naive group or vehicle group and the different treatment conditions were then compared. A typical HES-1 standard curve contained the following amounts of DNA: 2, 0.6, 0.2, 0.08, 0.02, 0.008, 0.002, 0.0007 pg DNA. A typical GAPDH standard curve contained the following amounts of DNA: 50, 16.67, 5.56, 1.85, 0.62, 0.21, 0.07, 0.02 pg DNA.

A quality control was run on all plates. The quality control was a large batch of cDNA prepared from a previously-run experiment. Sample naïve-1 from this experiment was used as a quality control sample.

Primer/probe sets used in Example 1 were purchased from Biosearch Technologies, of Novato, Calif. and are shown in Table 1.

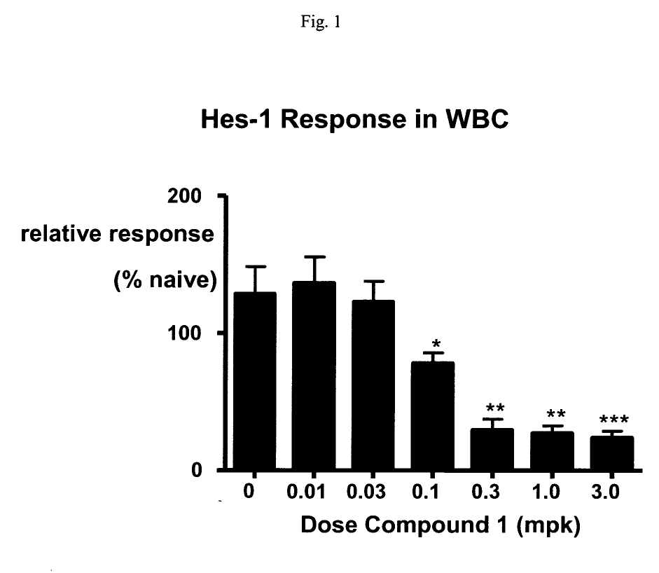

The results of Example 1 are shown below and in FIG. 1.

TABLE I

Primer Probe Sets for Detection of HES-1 and GAPDH
mRNA in Rat, Dog and Human WBCs Used in Example 1

| | |
|---|---|
| Rat Primer/Probe Info | |
| HES-1 | |
| Forward- | |
| 5′-TACCCCAGCCAGTGTCAACA-3′ | SEQ ID NO:XX |
| Reverse | |
| 5′-TCCATGATAGGCTTTGATGACTTTC-3′ | SEQ ID NO:XX |
| Probe(Fam/BHQ) | |
| 5′-CCGGACAAACCAAAGACAGCCTCTGA-3′ | SEQ ID NO:XX |
| GAPDH | |
| Forward | |
| 5′-CTCAACTACATGGTCTACATGTT-3′ | SEQ ID NO:XX |
| Reverse | |
| 5′-GTAGACTCCACGACATACTCAGC-3′ | SEQ ID NO:XX |
| Probe(Fam/BHQ) | |
| 5′-CCCATCACCATCTTCCAGGAGCGAG-3′ | SEQ ID NO:XX |
| Dog Primer/Probe | |
| HES-1 | |
| Forward- | |
| 5′-TACCCCGGCCAGTGTCAACA-3′ | SEQ ID NO:XX |
| Reverse | |
| 5′-TCCATGATAGGCTTGGATGACTTTC-3′ | SEQ ID NO:XX |
| Probe(Fam/BHQ) | |
| 5′-CCGGATAAACCAAAGACAGCATCCGA-3′ | SEQ ID NO:XX |
| GAPDH | |
| Forward | |
| 5′-CTCAACTACATGGTGTACATGTTCCAGT-3′ | SEQ ID NO:XX |
| Reverse | |
| 5′-GTGGACTCCACAACATACTCAGCAC-3′ | SEQ ID NO:XX |
| Probe(Fam/BHQ) | |
| 5′-TCCATCTCCATCTTCCAGGAGCGAG-3′ | SEQ ID NO:XX |
| Human Primer/Probe | |
| HES-1 | |
| Forward | |
| 5′-GGACATTCTGGAAATGACAGTGAA-3′ | SEQ ID NO:XX |
| Reverse | |
| 5′-AGCGCAGCCGTCATCTG-3′ | SEQ ID NO:XX |
| Probe(Fam/BHQ) | |
| 5′-CGGAACCTGCAGCGG-3′ | SEQ ID NO:XX |

TABLE II
Structure and in vitro Activity of the γ-Secretase
Inhibitors Compound 1 and Compound 2
Shown below are the in vivo potencies for inhibition of
APP and Notch processing for Compound 1 and Compound 2.

| Compound | APP (nM) | Notch (nM) |
|---|---|---|
| Compound 1 | 0.81 | 2.2 |
| Compound 2 | 2.0 | 35.7 |

TABLE III
Changes in WBC HES-1 mRNA levels Correlate with GI Histopathology in Dog

| | | | | | | Relative WBC Hes-1 mRNA | | |
|---|---|---|---|---|---|---|---|---|
| | | | | | | Levels | | |
| Compound 2 Terminal Dog Study | | | | | | Pre- and terminal-bleeds | | |
| | | | | | Free Plasma | (GAPDH normalized) | | |
| Dog | | Dose | | Tox | drug (nM) | Pre | Term | Term/Pre |
| 1 | male | Veh | | normal | | 1.09 | 0.41 | 0.37 |
| 6 | male | Veh | | normal | | | 0.30 | |
| 7 | female | Veh | | normal | | 0.53 | 0.48 | 0.91 |
| 12 | female | Veh | | normal | | 0.55 | 0.39 | 0.71 |
| 3 | male | 3/2 | mpk | normal | 25 | | | |
| 5 | male | 3/2 | mpk | normal | 24 | 0.36 | 0.23 | 0.63 |
| 9 | female | 3/2 | mpk | normal | 27 | 0.49 | | |
| 11 | female | 3/2 | mpk | normal | 19 | 0.59 | 0.44 | 0.74 |
| 2 | male | 15/10 | mpk | trace | ˜200 | 0.38 | | |
| 4 | male | 15/10 | mpk | trace | 227 | 0.52 | 0.09 | 0.18 |
| 8 | female | 15/10 | mpk | minimal | 207 | | 0.04 | |
| 10 | female | 15/10 | mpk | min/mild | 275 | 0.82 | 0.05 | 0.06 |

### Example 2

Rats were dosed orally with vehicle or different concentrations of the γ-secretase inhibitor Compound 1 QD for 4 days. Animals were sacrificed 5 hours-post dose on day 4. Sections of the duodenum were fixed and stained using the periodic acid-Schiff technique, which stains glycoproteins, to reveal mucin levels which are an indicator of goblet cell hyperplasia.

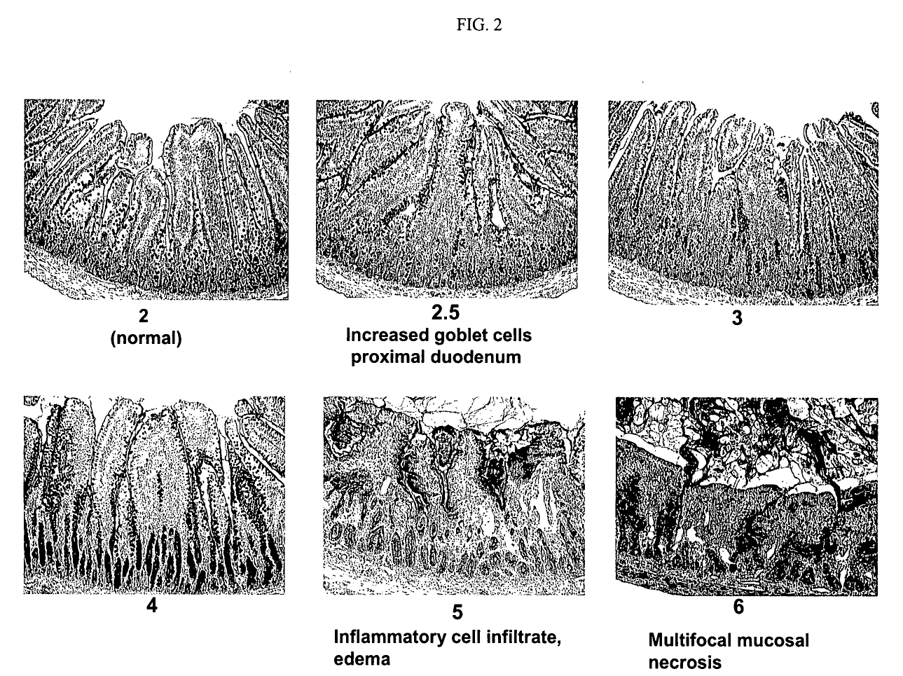

FIG. 2 depicts the results of this example. Summarily, this example demonstrates that γ-secretase inhibitors alter crypt stem cell differentiation in the small intestine.

### Example 3

Rats were dosed with vehicle or different concentrations of the γ-secretase inhibitor Compound 1 QD for 4 days. Animals were sacrificed on each of the 4 days, 5 hours-post dose. Sections of the duodenum were fixed and stained using the periodic acid-Schiff technique and the extent of goblet cell hyperplasia was determined.

Blood was collected by cardiac puncture and WBCs isolated by centrifugation.

WBC Hes-1 and GAPDH mRNA levels were measured by QRT-PCR and Hes-1 levels normalized to GAPDH. All values were normalized relative to naive, untreated control.

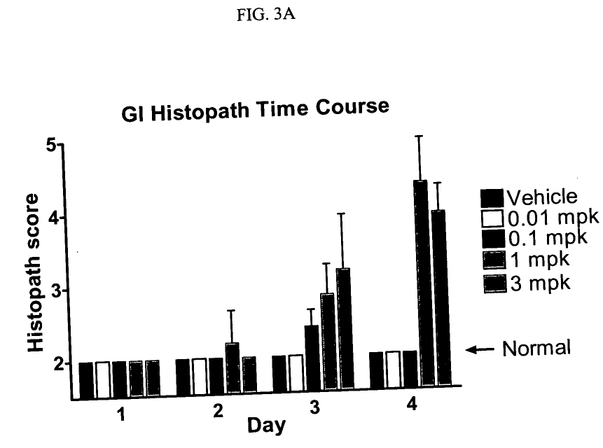

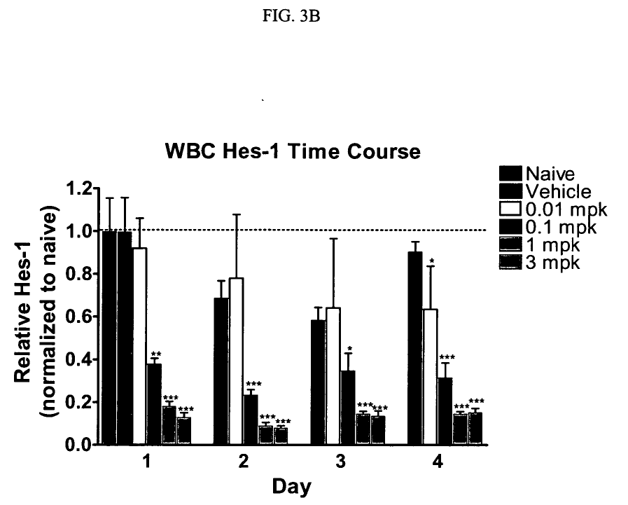

FIGS. 3A-3B depict the results of a representative experiment with mean±SD from 3 determinations indicated.

FIG. 3A depicts the full rat GI histopath time course.

FIG. 3B demonstrates that acute changes in WBC Hes-1 mRNA levels correlate with, and are predictive of, the GI histopathology observed on Days 3 and 4.

Summarily, Example 3 demonstrates acute changes in the WBC biomarker mRNA levels are predictive of GI histopathology observed on day 3 and day 4.

### References

The references cited in the specification are incorporated herein by reference to the extent that they supplement, explain, provide a background for or teach methodology, techniques and/or compositions employed herein. All publications and patents, including patent applications, referred to in this application are herein expressly incorporated by reference.

It will be understood that various details of the invention may be changed without departing from the scope of the invention. Furthermore, the foregoing description is for the purpose of illustration only.

1. A method of identifying a modulator of Notch processing in vivo mediated by γ-secretase comprising:
(a) determining an amount of an indicator in a sample comprising leukocytes acquired from a query subject in the presence and absence of the test compound; and
(b) comparing the amount of indicator acquired from the query subject in the presence of the test compound with an amount of indicator acquired from the query subject in the absence of the test compound;
wherein a change in the amount of indicator acquired in the presence of the test compound, compared with the amount of indicator acquired in absence of the test compound, indicates the compound modulates Notch processing mediated by γ-secretase activity.

2. The method of claim 1, wherein the indicator is selected from the group consisting of a Notch-regulated transcription factor, a Notch-regulated cell surface receptor, a Notch ligand, a Notch-regulated secreted factor, Hes-1, TCF3, SLC11A1, CD14, TRL4, CSPG and IL10.

3. The method of claim 1, wherein the leukocytes are lymphocytes or T cells.

4. The method of claim 1, where the query subject is selected from the group consisting of mice, rats, dogs, guinea pigs and humans.

5. The method of claim 1, wherein the step of determining the amount of the indicator comprises determining an amount of mRNA encoding the indicator present in the sample or comprises employing an analytical technique selected from the group consisting of Western blot, ELISA, RIA, quantitative real-time PCR, fluorescence activated cell sorting (FACs) and immunohistochemistry.

6. The method of claim 1, wherein the method is employed in a high-throughput operation or performed in a clinical trial.

7. The method of claim 1, further comprising repeating the method for each of a plurality of different test compounds.

8. A method of identifying a preferred dose of a test compound known or suspected to modulate Notch processing in vivo mediated by γ-secretase comprising:
(a) determining an amount of an indicator in a sample comprising leukocytes acquired from a query subject in the absence of the test compound;
(b) determining an amount of indicator and a Notch-related toxicity level in a sample comprising leukocytes acquired from a query subject in the presence of a first dose of the compound;
(c) repeating step (b) a for a plurality of different test compound doses;
(d) comparing
(i) the indicator amount; and
(ii) the Notch-related toxicity acquired in the presence of two or more doses of the test compound; and
(e) identifying a preferred dose of a compound known or suspected to modulate Notch processing mediated by γ-secretase based on an analysis of the comparison.

9. The method of claim 8, wherein the indicator is selected from the group consisting of a Notch-regulated transcription factor, a Notch-regulated cell surface receptor, a Notch ligand, a Notch-regulated secreted factor, Hes-1, TCF3, SLC11A1, CD14, TRL4, IL10 and CSPG.

10. The method of claim 8, wherein the leukocytes are lymphocytes or T cells.

11. The method of claim 8, where the query subject is selected from the group consisting of mice, rats, dogs, guinea pigs and humans.

12. The method of claim 8, wherein the step of determining an amount of the indicator comprises determining an amount of mRNA encoding the indicator present in the sample or comprises employing an analytical technique selected from the group consisting of Western blot, ELISA, RIA, quantitative real-time PCR, fluorescence activated cell sorting (FACs) and immunohistochemistry.

13. The method of claim 8, wherein Notch-related toxicity is determined by a technique selected from the group consisting of examining the immunohistochemistry of tissue sections and examining the morphology of goblet cells.

14. The method of claim 8, wherein the Notch-related toxicity is GI toxicity or goblet cell hyperplasia

15. The method of claim 8, wherein the method is employed in a high-throughput operation or in a clinical trial.

16. The method of claim 8, further comprising repeating the method for each of a plurality of different test compounds.

17. A method of generating a dosing schedule for a test compound known or suspected to modulate an activity mediated by γ-secretase comprising:
(a) determining an amount of indicator in a sample comprising leukocytes acquired from a query subject in the absence of the test compound;
(b) determining an amount of indicator in a sample comprising leukocytes acquired from the query subject in the presence of a first dose of the test compound at multiple time points;
(c) repeating step (b) for one or more doses of the test compound
(d) determining the Notch-related toxicity acquired in the presence of two or more doses of the test compound; and
(e) generating a dosing schedule based on a comparison of the observed indicator amounts and pharmacodynamics and associated Notch-related toxicity.

18. The method of claim 17, wherein the indicator is selected from the group consisting of a Notch-regulated transcription factor, a Notch-regulated cell surface receptor, a Notch ligand, a Notch-regulated secreted factor, Hes-1, Hes-5, Notch-1, Notch-2, Notch-3, Notch-4, Delta-1, Delta-3, Delta-4, Jagged-1, Jagged-2 and IL2.

19. The method of claim 17, wherein the leukocytes are lymphocytes or T cells.

20. The method of claim 17, where the query subject is selected from the group consisting of mice, rats, dogs, guinea pigs and humans.

21. The method of claim 17, wherein the step of determining an amount of the indicator comprises determining an amount of mRNA encoding the indicator present in the sample or comprises employing an analytical technique selected from the group consisting of Western blot, ELISA, RIA, quantitative real-time PCR, fluorescence activated cell sorting (FACs) and immunohistochemistry.

22. The method of claim 17, wherein Notch-related toxicity is determined by a technique selected from the group consisting of examining the immunohistochemistry of tissue sections and examining the morphology of goblet cells.

23. The method of claim 17, wherein the Notch-related toxicity is GI toxicity or goblet cell hyperplasia

24. The method of claim 17, wherein the method is employed in a high-throughput operation or in a clinical trial.

25. The method of claim 17, further comprising repeating the method for each of a plurality of different test compounds.

26. The method of claim 17, further comprising monitoring the dosing schedule.
